(19) United States
(12) Patent Application Publication
(10) Pub. No.: US 2026/0237482 A1
(43) Pub. Date: Aug. 13, 2026

(54) SYSTEM AND METHOD FOR GENERATING AND DELIVERING PATIENT-SPECIFIC MEDICATION INSTRUCTIONS

(71) Applicant: Tvisha Jagannathan, Monroe, NJ (US)

(72) Inventor: Tvisha Jagannathan, Monroe, NJ (US)

(21) Appl. No.: 19/646,472

(22) Filed: Apr. 13, 2026

(51) Int. Cl. G16H 20/10; G16H 70/40

(52) U.S. Cl. CPC G16H 20/10; G16H 70/40

(57) ABSTRACT

A system and method for generating and delivering patient-specific medication instructions are disclosed. Structured prescription data associated with a medication order is retrieved from an Electronic Health Record (EHR) data source in response to a request initiated through a user device. Personally identifiable information in the retrieved prescription data is masked to generate sanitized prescription data. Accessibility parameters associated with a patient profile, including preferred language, literacy level, visual impairment, dyslexia, and audio preference indicators, are retrieved. Medication instruction content is extracted from the sanitized prescription data and transformed into a normalized semantic representation comprising standardized medication instruction attributes. Patient-specific medication instructions are generated from the normalized semantic representation using artificial-intelligence-based constrained language generation engines. The generated instructions are rendered on the user device in various output formats, including audio output generated using a text-to-speech synthesis engine.

### BACKGROUND

### Technical Field

The present disclosure generally relates to healthcare information systems and digital health communication technologies. More particularly, the disclosure relates to systems and methods for generating and delivering patient-specific medication instructions based on prescription data and patient profile information.

### Description Of The Related Art

Medication instructions, including dosage schedules, administration routes, contraindications, and safety precautions, are essential for ensuring that patients properly understand and follow prescribed treatment regimens. In many healthcare systems, such instructions are typically provided to patients in the form of printed medication leaflets or labels generated at the point of dispensing. These instructions often originate from pharmacy dispensing systems or electronic health record (EHR) systems and are delivered in a standard textual format.

However, conventional medication information delivery approaches present several limitations. In many instances, medication instructions are generated only in a default language, often English, and are written at a literacy level that may be difficult for many patients to understand. Patients who speak other languages, have limited literacy skills, or require accessibility accommodations may find it challenging to interpret the information provided. As a result, patients may misunderstand dosage instructions, fail to recognize important warnings, or improperly administer medications.

Additionally, traditional medication instruction leaflets are primarily static and text-based. While some healthcare providers offer translated versions of instructions or provide large-print formats for certain patients, these solutions are typically generated manually or through simple text-translation systems and do not account for patient-specific needs such as literacy level, sensory impairments, or preferred delivery format. Furthermore, conventional systems rarely provide coordinated delivery of medication instructions across multiple modalities, such as audio, video, or pictographic representations that may improve patient comprehension.

Another limitation of existing approaches is the lack of integration between medication instruction generation systems and patient profile data stored in healthcare information systems. In many cases, patient accessibility preferences, language requirements, or other personalization parameters are not dynamically incorporated into the generation of medication instructions. Consequently, instructions are often generic and not tailored to the specific needs of individual patients.

Moreover, existing medication information systems generally do not support automated updates when prescription details change. Modifications to prescription parameters, such as dosage adjustments, formulation changes, or newly identified contraindications, may not be promptly reflected in previously generated patient instructions. This may lead to outdated guidance being relied upon by patients.

Accordingly, there exists a need for an improved system and method for generating and delivering patient-specific medication instructions that account for prescription information, patient accessibility parameters, and language preferences while supporting multiple output formats and updated instruction delivery when prescription information changes.

The reference to any prior art in this specification is not an acknowledgment or suggestion that prior art forms part of the common general knowledge in any jurisdiction or that a person skilled in the art could reasonably expect that prior art to be understood, regarded as relevant, and/or combined with other aspects of the prior art.

### BRIEF SUMMARY

One or more embodiments of the present disclosure provide a system and method for generating and delivering patient-specific medication instructions based on prescription data and patient profile information. The system retrieves structured prescription data associated with a medication order from a healthcare data source and processes the data to produce instructions tailored to an individual patient. The prescription data is first sanitized to remove personally identifiable information. Medication instruction content is then extracted and transformed into a structured representation capturing key prescription details such as dosage, administration route, duration, contraindications, and warnings. Using this representation, an artificial intelligence-based language generation engine, based on retrieved accessibility parameters, generates patient-specific medication instructions that may incorporate parameters from the patient profile, including language preference and accessibility requirements.

In an embodiment, the generated instructions are rendered in one or more output formats. In one implementation, the instructions are provided as audio output using a text-to-speech synthesis process. Additional formats may include text, video, or pictographic representations to support improved patient comprehension. In some implementations, access to the generated instructions is provided through a machine-readable encoded access object, such as a QR code, near-field communication element, or embossed scannable label, which references the instructions. The encoded access object may be generated using an encrypted reference identifier, including an expiration parameter. In certain embodiments, the pictographic graphical output and visual representations may be generated in accordance with standardized healthcare communication guidelines, including internationally recognized medical pictogram standards, to improve comprehension across diverse patient populations.

An embodiment of the present disclosure provides a system for generating and delivering patient-specific medication instructions based on prescription data and patient profile information. The system includes a plurality of processing modules configured to retrieve prescription data, process the data to generate patient-specific instructions, and deliver the instructions in accessible formats. The structured prescription data is retrieved in response to a request initiated by a user through a user device.

In an embodiment, the system includes a data retrieval module configured to retrieve structured prescription data associated with a medication order for a patient profile from an Electronic Health Record (EHR) data source. The structured prescription data may include prescription attributes such as dosage, administration route, frequency, duration, contraindications, and warnings.

In an embodiment, the system further includes a data sanitization module configured to mask personally identifiable information present in the structured prescription data to generate sanitized prescription data without personally identifiable information. In one implementation, the data sanitization module masks personally identifiable information by replacing personally identifiable information fields in the structured prescription data with anonymized tokens to generate the sanitized prescription data.

In an embodiment, the system includes an instruction extraction module configured to extract medication instruction content from the sanitized prescription data. The instruction extraction module may perform structured data parsing of prescription fields, including dosage, administration route, frequency, duration, contraindications, and warning information to obtain relevant medication instruction content.

In an embodiment, the system includes a semantic transformation module configured to transform the medication instruction content into a normalized semantic representation. The normalized semantic representation may comprise a standardized semantic medication instruction object including attributes such as dosage frequency, administration route, duration, contraindications, and warning attributes.

In an embodiment, the system further includes a patient profile retrieval module configured to retrieve accessibility parameters associated with the patient profile. The accessibility parameters may include at least one of a preferred language, a literacy level indicator, a visual impairment indicator, a dyslexia indicator, and an audio-preference indicator.

In an embodiment, the system includes an instruction generation module configured to generate patient-specific medication instructions, including textual instructions in the preferred languages and corresponding speech representations, from the normalized semantic representation using artificial-intelligence-based constrained language generation engines based on accessibility parameters associated with the patient profile. The artificial-intelligence-based constrained language generation engines may perform one or more operations including applying language translation consistent with the preferred language, applying literacy-level simplification rules based on the literacy level indicator, adapting the generated patient-specific medication instructions based on the visual impairment indicator, adapting the generated instructions based on the dyslexia indicator, adapting the generated instructions based on the audio-preference indicator, and generating the instructions using a dialect variant of the preferred language determined from regional parameters stored in the patient profile.

In an embodiment, the system includes a rendering module configured to render the generated patient-specific medication instructions on the user device in at least one output format. In one implementation, the output format comprises audio output generated by a text-to-speech synthesis engine configured to convert the generated patient-specific medication instructions into spoken instructions using phonetic conversion and speech synthesis models. In an embodiment, the output format may further comprise text output generated by converting the generated patient-specific medication instructions into formatted instructional text using a structured medical instruction template. The text output may be rendered in a font size determined based on the visual impairment indicator associated with the patient profile. In an embodiment, the output format may further comprise video output generated by a video composition engine configured to assemble a sequence of instructional visual segments based on prescription dosage parameters. The video composition engine may map dosage timing parameters to a temporal sequence of animated visual scenes representing medication administration events. In certain implementations, the system may further include an audiovisual synchronization controller configured to synchronize synthesized speech with animated graphical overlays.

In an embodiment, the output format may further comprise pictographic graphical output generated by mapping medication administration parameters to a predefined library of dosage and administration pictograms. In an embodiment, the output format may comprise a machine-readable encoded access object embedding a reference to the generated patient-specific medication instructions. The machine-readable encoded access object may include at least one of a QR code, a near-field communication element, or an embossed scannable label.

In an embodiment, the machine-readable encoded access object may be generated using an encrypted reference identifier, wherein the encrypted reference identifier is encoded into the machine-readable encoded access object and includes an expiration parameter limiting access validity to a predefined duration.

In an embodiment, the system includes an update listener module configured to detect modification of at least one prescription parameter in the EHR data source. The detected modification may include a dosage modification, a medication formulation change, an added contraindication warning, or a medication discontinuation event. In an embodiment, detection of the modification by the update listener module triggers regeneration of the patient-specific medication instructions and invalidation of previously generated machine-readable encoded access objects to ensure that patients receive updated medication guidance.

An embodiment of the present disclosure provides a method for generating and delivering patient-specific medication instructions. The method includes retrieving, from an Electronic Health Record (EHR) data source, structured prescription data associated with a medication order for a patient profile. The structured prescription data is retrieved in response to a request initiated by a user through a user device. Further, the method includes masking personally identifiable information in the structured prescription data to generate sanitized prescription data without personally identifiable information. Furthermore, the method includes retrieving accessibility parameters associated with the patient profile. The accessibility parameters include a preferred language, a literacy level indicator, a visual impairment indicator, a dyslexia indicator, and an audio-preference indicator.

In an embodiment, the method includes extracting medication instruction content from the sanitized prescription data. Further, the method includes transforming the medication instruction content into a normalized semantic representation. The normalized semantic representation includes a standardized semantic medication instruction object. The standardized semantic medication instruction object includes dosage frequency, administration route, duration, contraindications, and warning attributes. In an embodiment, the method includes generating patient-specific medication instructions from the normalized semantic representation using artificial-intelligence-based constrained language generation engines based on accessibility parameters associated with the patient profile.

In an embodiment, the method includes rendering the generated patient-specific medication instructions in at least one output format. The output format comprises audio output generated by a text-to-speech synthesis engine configured to convert the generated patient-specific medication instructions into spoken instructions using phonetic conversion and speech synthesis models.

The features and advantages of the subject matter herein will become more apparent in light of the following detailed description of selected embodiments, as illustrated in the accompanying FIGUREs. As will be realized, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label, irrespective of the second reference label.

FIG. 1 illustrates an environment having a system for generating and delivering patient-specific medication instructions, in accordance with an embodiment of the present disclosure.

FIG. 2 illustrates a block diagram of the system for generating and delivering patient-specific medication instructions, in accordance with an embodiment of the present disclosure.

FIG. 3A-3B illustrates exemplary multimedia medication instruction interfaces presented on a user device for displaying patient-specific medication instructions, in accordance with an embodiment of the present disclosure.

FIG. 4 illustrates an exemplary interface presented on the user device for enabling the retrieval of patient-specific medication instructions through the machine-readable encoded access object, in accordance with an embodiment of the present disclosure.

FIG. 5 illustrates an exemplary flowchart of a method for generating and delivering patient-specific medication instructions, in accordance with an embodiment of the present disclosure.

FIG. 6 illustrates an exemplary computer system in which or with which embodiments of the present disclosure may be utilized.

Other features of embodiments of the present disclosure will be apparent from accompanying drawings and detailed description that follows.

### DETAILED DESCRIPTION

### Terminology

Brief definitions of terms used throughout this application are given below.

The terms “connected” or “coupled”, and related terms, are used in an operational sense and are not necessarily limited to a direct connection or coupling. Thus, for example, two devices may be coupled directly, or via one or more intermediary media or devices. As another example, devices may be coupled in such a way that information can be passed there between, while not sharing any physical connection with one another. Based on the disclosure provided herein, one of ordinary skill in the art will appreciate a variety of ways in which connection or coupling exists in accordance with the aforementioned definition.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

Exemplary embodiments will now be described more fully hereinafter with reference to the accompanying drawings, in which exemplary embodiments are shown. This disclosure may, however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Certain exemplary embodiments of the present invention are described below and illustrated in the accompanying figures. The embodiments described are only for purposes of illustrating the present invention and should not be interpreted as limiting the scope of the invention, which, of course, is limited only by the claims below. Other embodiments of the invention, and certain modifications and improvements of the described embodiments, will occur to those skilled in the art, and all such alternate embodiments, modifications, and improvements are within the scope of the present invention.

According to common practice, the various features of the drawings discussed below are not necessarily drawn to scale. Dimensions of various features and elements in the drawings may be expanded or reduced to more clearly illustrate the embodiments of the invention.

The present disclosure provides a system and method for generating and delivering patient-specific medication instructions based on prescription data and patient profile information. The system retrieves prescription data associated with a patient from a healthcare data source and processes the data to generate instructions tailored to the patient's needs. In an embodiment, prescription data is first processed to remove personally identifiable information, after which relevant medication instruction content is extracted. The extracted content is transformed into a structured representation that enables accurate and consistent interpretation of prescription details such as dosage, administration route, duration, contraindications, and warnings. Using this information, the system generates patient-specific medication instructions through an artificial intelligence-based language generation engine. The generation process may incorporate patient profile parameters, including language preference, literacy level, and accessibility requirements, thereby producing instructions that are easier for the patient to understand and follow.

In an embodiment, the generated instructions are then rendered in one or more output formats. In one implementation, the instructions are converted into spoken instructions using a text-to-speech synthesis process, enabling patients to receive medication guidance in audio form. Additional output formats may include text instructions, video-based explanations, or pictographic representations designed to improve comprehension for patients with different accessibility needs. In certain embodiments, such pictographic representations may be aligned with standardized healthcare pictogram libraries and accessibility guidelines to ensure consistent interpretation of medication instructions across different patient populations.

In certain embodiments, the system may provide access to the generated instructions through a machine-readable encoded access object, such as a QR code, near-field communication element, or embossed scannable label. The encoded object may reference the generated instructions and allow patients to access the information using compatible devices. The encoded access object may incorporate security features such as encrypted reference identifiers and expiration parameters.

In an embodiment, the system may also monitor prescription information for updates. When modifications to prescription parameters are detected, such as dosage changes or newly added warnings, the system may regenerate the patient-specific instructions and invalidate previously generated access objects to ensure that patients receive current guidance. By combining automated generation of patient-specific instructions with flexible delivery formats and update mechanisms, the disclosed system improves the ability of patients to access and understand medication information relevant to their prescribed treatment.

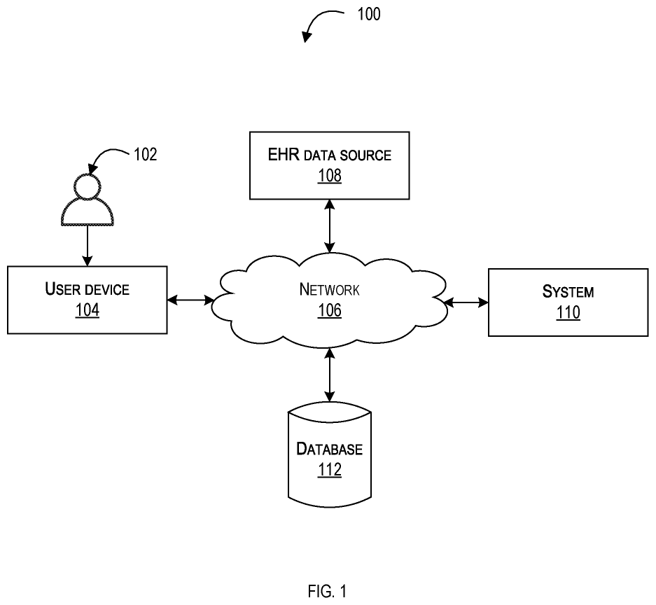

FIG. 1 illustrates an environment 100 having a system 110 for generating and delivering patient-specific medication instructions, in accordance with an embodiment of the present disclosure. In an embodiment, the environment 100 may include a user 102, a user device 104, a communication network 106, an Electronic Health Record (EHR) data source 108, the system 110, and a database 112.

In an embodiment, the user 102 may correspond to an individual associated with a medication prescription for whom medication instructions are generated and delivered by the system 110. The user 102 may represent a patient who has been prescribed one or more medications by a healthcare provider. The user 102 may access the medication instructions using one or more output formats generated by the system 110. In an embodiment, the user 102 may also include caregivers, family members, pharmacists, healthcare practitioners, or other authorized individuals who may access medication instructions on behalf of the patient. For example, a pharmacist may initiate the generation of patient-specific medication instructions when dispensing medication, while a caregiver may access the instructions to assist the patient in following the prescribed medication regimen.

In some embodiments, the patient profile associated with the user 102 may include accessibility parameters that influence how medication instructions are generated and delivered. Such accessibility parameters may include a preferred language, a literacy level indicator, a visual impairment indicator, a dyslexia indicator, and an audio-preference indicator. These parameters may enable the system 110 to generate instructions that are understandable and accessible to the user 102. In an embodiment, the user 102 may interact with the system 110 indirectly through the user device 104.

In an embodiment, the user device 104 may be configured to enable the user 102 to access medication instructions generated by the system 110 and delivered in one or more output formats. In some embodiments, the user 102 may initiate generation of the medication instructions by providing a request through the user device 104, for example, by selecting a generate instruction option, issuing a command, or otherwise requesting retrieval of medication instructions associated with a medication order corresponding to a patient profile. In response to the request, the system 110 may retrieve prescription data and generate patient-specific medication instructions accessible through the user device 104.

In an embodiment, the user device 104 may include any computing device capable of receiving, processing, displaying, or rendering digital content. Examples of the user device 104 may include, but are not limited to, a smartphone, a tablet computing device, a laptop computer, a desktop computer, a wearable computing device, or any other electronic device capable of executing application software and communicating with remote systems through the communication network 106.

In an embodiment, the user device 104 may include one or more input/output interfaces configured to facilitate the user 102 to interact with generated medication instructions. Such interfaces may include a display interface for presenting text instructions, graphical content, pictograms, or video instructions. The user device 104 may also include audio output components, such as speakers or headphones, configured to render spoken medication instructions generated by the system 110.

In an embodiment, the user device 104 may execute one or more applications or web-based interfaces configured to access instructions generated by the system 110. For example, the user device 104 may access an audio playback interface, a video playback interface, or a graphical instruction viewer that presents medication guidance in formats adapted to the accessibility parameters of the user 102.

In certain implementations, the user device 104 may communicate with the system 110 through the communication network 106 to request, retrieve, or stream generated medication instructions. The communication network 106 may facilitate communication between the user device 104, the EHR data source 108, the system 110, and the database 112. The communication network 106 may enable transmission of prescription data, patient profile information, generated medication instructions, and associated references between the components of the environment 100.

In an embodiment, the communication network 106 may include one or more wired or wireless communication networks. Examples of the communication network 106 may include, but are not limited to, the Internet, a local area network (LAN), a wide area network (WAN), a metropolitan area network (MAN), a cellular communication network, a wireless fidelity (Wi-Fi) network, or any combination thereof.

In some embodiments, the communication network 106 may support communication protocols that enable secure exchange of healthcare information between systems. Such protocols may include, for example, Hypertext Transfer Protocol (HTTP), Hypertext Transfer Protocol Secure (HTTPS), Transmission Control Protocol/Internet Protocol (TCP/IP), or other data communication protocols used in healthcare information systems. In an embodiment, the communication network 106 may enable the system 110 to retrieve structured prescription data from the EHR data source 108 and to obtain patient profile information relevant to generating patient-specific medication instructions. The communication network 106 may also allow the system 110 to transmit references to generated medication instructions to the user device 104.

In some embodiments, the communication network 106 may support both real-time communication and asynchronous communication between system components. Real-time communication may be used when retrieving prescription updates or generating instructions during medication dispensing, while asynchronous communication may occur when the user 102 accesses the instructions at a later time through the user device 104.

In an embodiment, the EHR data source 108 may store and provide access to healthcare-related data associated with the user 102. In particular, the EHR data source 108 may maintain prescription data, patient profile information, and other clinical information relevant to medication management. In an embodiment, the EHR data source 108 may include one or more healthcare information systems that maintain electronic records of patient health information. Such systems may include electronic health record systems, electronic medical record systems, pharmacy dispensing systems, clinical information systems, or other healthcare data repositories used by healthcare providers and pharmacies. In certain embodiments, the EHR data source 108 may operate within regulated healthcare information environments that enforce compliance with healthcare privacy, security, and clinical information management regulations governing the storage and transmission of patient-related data.

In an embodiment, the EHR data source 108 may store structured prescription data associated with medication orders prescribed to the user 102. The structured prescription data may include, but is not limited to, medication identifiers, dosage information, administration routes, frequency schedules, duration of treatment, contraindications, warnings, and other prescription attributes associated with a medication order.

In some embodiments, the EHR data source 108 may also store patient profile information corresponding to the user 102. The patient profile information may include patient identifiers, demographic attributes, and accessibility parameters that may be used by the system 110 when generating medication instructions. Such accessibility parameters may include, for example, a preferred language, a literacy level indicator, a visual impairment indicator, a dyslexia indicator, and an audio-preference indicator.

In an embodiment, the system 110 may retrieve structured prescription data and patient profile information from the EHR data source 108 through the communication network 106. The retrieved prescription data may correspond to medication orders issued by healthcare providers and processed by pharmacy systems when medications are dispensed. In some embodiments, the EHR data source 108 may include multiple distributed healthcare data repositories that collectively provide prescription information and patient profile data used by the system 110.

In an embodiment, the database 112 may be communicatively coupled to the system 110. The database 112 may be configured to store data used by the system 110 during the generation, processing, and delivery of patient-specific medication instructions. In an embodiment, the database 112 may include one or more data storage repositories capable of storing structured and unstructured data. The database 112 may be implemented using relational databases, non-relational databases, distributed storage systems, or cloud-based storage infrastructure.

In an embodiment, the database 112 may store intermediate and processed data generated during operation of the system 110. In an embodiment, the database 112 may be maintained within the infrastructure of the system 110 or may be implemented as an external storage service accessible through the communication network 106. The database 112 may support data retrieval and storage operations performed by the system 110 during execution of the processes described herein.

In operation, the user 102 may initiate the request through the user device 104 to obtain medication instructions associated with a medication order corresponding to the patient profile. In response to the request, the system 110 may retrieve structured prescription data associated with the medication order for the user 102 from the EHR data source 108 through the communication network 106. The retrieved prescription data may include information related to medication dosage, administration route, frequency, duration, contraindications, and warnings.

In certain embodiments, generation of patient-specific medication instructions may be automatically triggered during pharmacy dispensing operations. For example, when a pharmacy dispensing system processes a medication order associated with the patient profile, the dispensing system may transmit a request to the system 110 to generate personalized medication instructions corresponding to the dispensed medication. The generated instructions may then be incorporated into medication leaflets, labels, or machine-readable encoded access objects provided with the dispensed medication.

In an embodiment, the system 110 may also obtain patient profile information corresponding to the user 102. The system 110 may process the retrieved prescription data and generate medication instructions tailored to the patient profile associated with the user 102. In an embodiment, the generated medication instructions may be rendered in one or more output formats accessible to the user 102. For example, the instructions may be provided in audio form, textual form, video-based guidance, or pictographic representations. The user 102, using the user device 104, may access the generated medication instructions through the communication network 106.

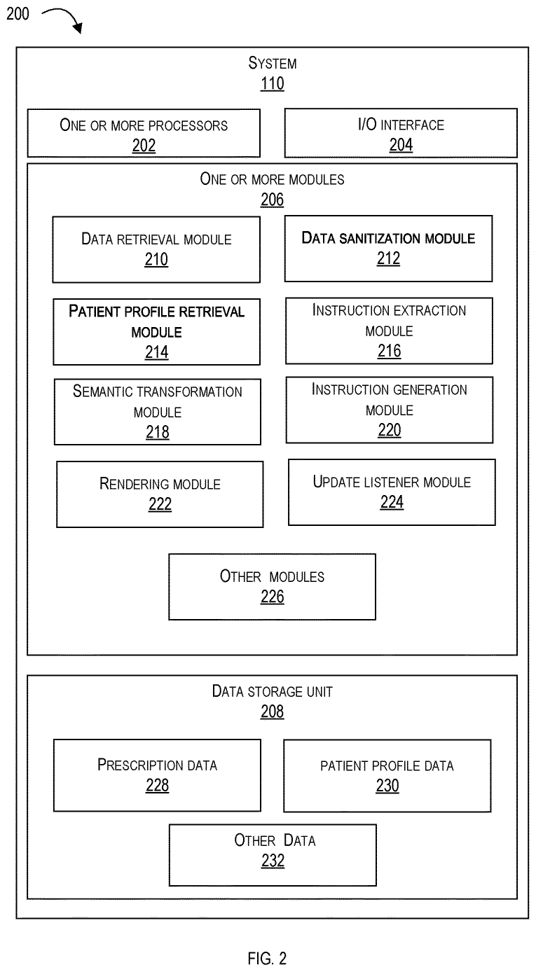

FIG. 2 illustrates a block diagram 200 of the system 110 for generating and delivering patient-specific medication instructions, in accordance with an embodiment of the present disclosure. In an embodiment, the system 110 may include one or more processors 202, an Input/Output (I/O) interface 204, one or more modules 206, and a data storage unit 208. The one or more processors 202 may be implemented as microprocessors, microcontrollers, central processing units, programmable logic circuitry, application-specific integrated circuits, or other computing components configured to execute computer-readable instructions.

In an embodiment, the processors 202 may be configured to execute instructions stored in memory to manage the overall operation of the system 110. Such operations may include retrieving structured prescription data associated with a medication order from an Electronic Health Record (EHR) data source 108, processing prescription information, generating patient-specific medication instructions based on prescription attributes and patient profile parameters, rendering the generated instructions in one or more accessible output formats, and enabling delivery of the instructions through machine-readable encoded access objects or other communication mechanisms.

The I/O interface 204 may facilitate communication between internal components of the system 110 and external systems. Such external systems may include user devices 104, EHR data sources 108, communication networks 106, databases, pharmacy dispensing systems, healthcare information systems, or other computing systems capable of exchanging healthcare-related data. In certain embodiments, the I/O interface 204 may support wired or wireless communication protocols to enable transmission and reception of prescription data, patient profile information, generated medication instructions, encoded access object references, and related healthcare information. Such external systems may include pharmacy dispensing systems, electronic health record systems operating within regulated healthcare environments that enforce healthcare privacy, security, and data integrity standards.

In one or more embodiments, the one or more modules 206 may include, without limitation, a data retrieval module 210, a data sanitization module 212, a patient profile retrieval module 214, an instruction extraction module 216, a semantic transformation module 218, an instruction generation module 220, a rendering module 222, an update listener module 224, and other module(s) 226. The modules 206 may be implemented in hardware, software, firmware, or any combination thereof, and may be communicatively coupled to the processors 202.

The data storage unit 208 may be implemented using persistent memory, non-volatile memory, flash storage, magnetic storage, or any computer-readable storage medium accessible to the processors 202. The data storage unit 208 may store prescription data 228, patient profile data 230, and other data 232 associated with prescription attributes, accessibility parameters, multimedia instruction content, and system-generated outputs used during operation of the system 110.

In an embodiment, the data retrieval module 210 may retrieve structured prescription data associated with a medication order for a patient profile from an Electronic Health Record (EHR) data source 108. The data retrieval module 210 may obtain prescription data corresponding to medications prescribed to the user 102 by a healthcare provider. In certain embodiments, the retrieval of the structured prescription data may occur in response to a request initiated by the user 102 through the user device 104, for example, when the user requests the generation of medication instructions associated with a medication order. The structured prescription data may include, but is not limited to, medication identifiers, dosage information, administration routes, frequency schedules, treatment duration, contraindications, warnings, and other prescription attributes associated with a medication order.

In an embodiment, the data retrieval module 210 may retrieve prescription data through Application Programming Interfaces (APIs), healthcare interoperability interfaces, or other data exchange mechanisms provided by healthcare information systems. For example, the EHR data source 108 may expose standardized healthcare data interfaces through which prescription records associatedchatgpt with the patient profile may be accessed.

In some embodiments, the structured prescription data retrieved by the data retrieval module 210 may be represented in standardized healthcare data formats. Such formats may include structured prescription records generated by pharmacy dispensing systems, clinical information systems, electronic medical record systems, or other healthcare data management platforms. The retrieved prescription data may be stored temporarily in data storage unit 208 or within the database 112 to enable subsequent processing by other components of the system 110.

In an embodiment, the data retrieval module 210 may retrieve prescription data corresponding to newly issued medication orders, updated prescriptions, or existing prescription records associated with the patient profile. The retrieval operation may occur in response to triggering events, including prescription issuance, medication dispensing events, updates to prescription parameters, or requests initiated by healthcare providers, pharmacists, or other authorized systems. In some implementations, the retrieval may also be initiated by the user 102 through the user device 104 when the user requests the generation or retrieval of medication instructions associated with the medication order. In certain embodiments, retrieval of the structured prescription data may also be triggered automatically during pharmacy dispensing operations. For example, when a pharmacy dispensing system processes a medication order corresponding to the patient profile, the dispensing system may transmit a request to the system 110 to initiate the generation of patient-specific medication instructions.

In an embodiment, the data retrieval module 210 may also support the retrieval of historical prescription records associated with the patient profile. Historical prescription information may be used to determine changes in medication instructions over time, detect prescription modifications, or support the generation of updated patient-specific medication instructions when prescription parameters are modified. In certain embodiments, the data retrieval module 210 may retrieve prescription data from multiple healthcare data repositories. For example, prescription information may be retrieved from an EHR system maintained by a healthcare provider, while dispensing information may be retrieved from a pharmacy dispensing system. The data retrieval module 210 may aggregate such data to produce a consolidated representation of prescription information associated with the patient profile.

In some embodiments, the data retrieval module 210 may also support real-time or near real-time retrieval of prescription updates. Such retrieval may allow the system 110 to obtain updated prescription parameters when modifications occur in the EHR data source 108. These modifications may include dosage changes, formulation changes, addition of contraindication warnings, and/or discontinuation of medications.

In an embodiment, the data sanitization module 212 may mask Personally Identifiable Information (PII) present in the structured prescription data to generate sanitized prescription data without personally identifiable information. The structured prescription data retrieved from the EHR data source 108 may include certain patient-identifying attributes such as patient identifiers, names, contact details, medical record numbers, or other information that may uniquely identify the user 102. The data sanitization module 212 may remove, mask, or replace such personally identifiable information prior to subsequent processing by other components of the system 110.

In an embodiment, the data sanitization module 212 may perform masking of personally identifiable information by replacing one or more personally identifiable fields with anonymized tokens. The anonymized tokens may serve as internal identifiers that allow the system 110 to associate prescription information with a corresponding patient profile while preventing exposure of sensitive patient identity information during subsequent processing operations. For example, patient names or identifiers may be replaced with system-generated tokens that preserve record linkage without revealing the original identifying information.

In certain implementations, the data sanitization module 212 may identify personally identifiable information fields within the structured prescription data using predefined field mappings or data schemas associated with healthcare data formats. For example, specific data fields representing patient identifiers, demographic attributes, or contact information may be recognized as sensitive fields that require masking or anonymization. The data sanitization module 212 may selectively remove or transform such fields while preserving prescription attributes required for generating medication instructions. In some embodiments, the data sanitization module 212 may support different levels of anonymization depending on system configuration or regulatory requirements. For example, certain implementations may completely remove patient-identifying fields, while other implementations may replace the fields with encrypted identifiers or pseudonymous tokens. Such approaches may allow the system 110 to maintain internal data associations while protecting patient privacy.

In certain embodiments, the data sanitization operations may be implemented in accordance with healthcare data privacy and security regulations governing handling of protected health information. For example, sanitization, anonymization, and tokenization operations performed by the data sanitization module 212 may comply with regulatory frameworks such as the Health Insurance Portability and Accountability Act (HIPAA) or other applicable healthcare privacy standards to ensure that sensitive patient information is protected during processing and instruction generation operations.

In an embodiment, the patient profile retrieval module 214 may retrieve accessibility parameters associated with the patient profile corresponding to the user 102. The accessibility parameters may represent attributes or preferences associated with the patient profile that influence how medication instructions are generated and delivered to the user 102. In an embodiment, the patient profile retrieval module 214 may obtain the accessibility parameters from the EHR data source 108 or from other healthcare information systems associated with the patient profile. The retrieved accessibility parameters may include, but are not limited to, a preferred language, a literacy level indicator, a visual impairment indicator, a dyslexia indicator, and an audio-preference indicator. These parameters may allow the system 110 to tailor generated medication instructions according to the accessibility requirements of the user 102.

In certain implementations, the preferred language parameter may indicate a language selected by the user 102 or recorded in the patient profile within the EHR data source 108. The preferred language parameter may be used to ensure that generated medication instructions are produced in a language understandable to the user 102. In some embodiments, the preferred language parameter may also include information related to dialect variants or regional language preferences. In some embodiments, the literacy level indicator may represent an estimate or classification of the reading comprehension level associated with the user 102. The literacy level indicator may be derived from patient profile information recorded by healthcare providers or inferred from accessibility settings associated with the patient profile. The literacy level indicator may be used to adjust the complexity, terminology, or phrasing used when generating medication instructions. In certain embodiments, the visual impairment indicator may represent information indicating that the user 102 may have difficulty reading standard text formats. For example, the visual impairment indicator may correspond to conditions such as reduced vision or other visual accessibility needs recorded within the patient profile. This parameter may influence how medication instructions are rendered or formatted for the user 102. In some embodiments, the dyslexia indicator may represent an accessibility parameter associated with reading difficulties experienced by the user 102. The dyslexia indicator may allow the system 110 to generate medication instructions that avoid complex sentence structures, reduce textual density, or incorporate alternative instructional formats to improve comprehension. In certain implementations, the audio-preference indicator may represent a preference of the user 102 for receiving medication instructions in audio form. For example, the patient profile may indicate that the user prefers spoken instructions rather than text-based instructions. This parameter may influence how the generated medication instructions are rendered and delivered to the user device 104.

In some embodiments, the patient profile retrieval module 214 may retrieve the accessibility parameters in response to the generation of medication instructions for a specific medication order associated with the patient profile. In other embodiments, the accessibility parameters may be retrieved in advance and stored temporarily within data storage unit 208 or within the database 112.

In an embodiment, the instruction extraction module 216 may extract medication instruction content from the sanitized prescription data. The sanitized prescription data may include structured prescription attributes associated with a medication order corresponding to the patient profile of the user 102. In an embodiment, the instruction extraction module 216 may identify and extract prescription fields representing medication instruction information required for generating patient-specific medication instructions. Such prescription fields may include, but are not limited to, dosage information, administration route, administration frequency, treatment duration, contraindications, warning information, and other medication administration parameters associated with the medication order.

In certain implementations, the instruction extraction module 216 may perform structured data parsing on the sanitized prescription data to isolate relevant instruction fields. For example, the instruction extraction module 216 may analyze structured prescription records obtained from pharmacy dispensing systems or electronic health record systems and extract fields corresponding to dosage schedules, medication intake instructions, and associated precautionary information. In some embodiments, the instruction extraction module 216 may operate using predefined parsing rules or data schemas associated with structured healthcare data formats. For example, the instruction extraction module 216 may recognize standardized prescription fields defined within electronic prescription records, clinical information systems, or pharmacy management systems. In an embodiment, the instruction extraction module 216 may map such fields into an internal representation suitable for further processing by the system 110.

In certain embodiments, the instruction extraction module 216 may also process prescription instructions represented in textual form within the structured prescription data. For example, prescription instructions entered by healthcare providers may include textual dosage instructions describing medication intake schedules. The instruction extraction module 216 may analyze such textual instructions and identify relevant medication administration parameters contained within the instructions. In some implementations, the instruction extraction module 216 may support the extraction of multiple medication instruction components associated with a single medication order. For example, a medication order may include dosage instructions for different times of day, specific intake conditions such as administration with food, or warnings related to potential side effects or contraindications. The instruction extraction module 216 may identify and extract such instruction components to ensure that the resulting instruction content accurately represents the prescribed medication regimen.

In certain embodiments, the instruction extraction module 216 may also support the extraction of additional contextual attributes associated with the medication instructions. Such attributes may include medication names, formulation details, administration methods, precautionary statements, or safety warnings associated with the medication order. In an embodiment, the instruction extraction module 216 may generate an intermediate representation of the extracted medication instruction content. The intermediate representation may include structured elements representing dosage frequency, administration route, treatment duration, contraindications, and warning attributes extracted from the sanitized prescription data.

In some embodiments, the instruction extraction module 216 may perform validation checks to ensure that the extracted medication instruction content includes the required prescription parameters needed for the generation of patient-specific medication instructions. For example, the module may verify that dosage instructions and administration schedules are present and properly structured. In an embodiment, the extracted medication instruction content generated by the instruction extraction module 216 may be provided to subsequent components of the system 110 for further processing. In particular, the extracted instruction content may be supplied to the semantic transformation module for conversion into a normalized semantic representation suitable for automated instruction generation.

In an embodiment, the semantic transformation module 218 may transform the medication instruction content extracted by the instruction extraction module 216 into a normalized semantic representation. The normalized semantic representation may provide a structured and standardized representation of the medication instruction content, thereby enabling consistent processing of prescription information during subsequent instruction generation operations. In an embodiment, the normalized semantic representation may comprise a standardized semantic medication instruction object representing prescription attributes associated with the medication order. The semantic medication instruction object may include structured elements representing at least one of dosage frequency, administration route, treatment duration, contraindications, warning attributes, and other medication administration parameters derived from the extracted medication instruction content.

In certain implementations, the semantic transformation module 218 may convert prescription fields extracted from the sanitized prescription data into standardized semantic attributes. For example, dosage instructions obtained from the prescription record may be mapped into normalized fields representing dosage quantity, dosage frequency, and timing of medication administration. Such normalization may allow prescription information originating from different healthcare systems to be represented using a consistent internal structure. In some embodiments, the semantic transformation module 218 may utilize predefined transformation rules, data models, or semantic mappings to convert extracted prescription attributes into the normalized semantic representation. The transformation rules may define how prescription fields retrieved from healthcare data systems are interpreted and mapped into standardized semantic elements used by the system 110. In certain embodiments, the semantic transformation module 218 may utilize standardized medical ontologies or pharmaceutical terminology standards to normalize prescription attributes. Such ontologies may include structured medical vocabularies or drug terminology databases used within healthcare systems to ensure consistency and regulatory compliance of clinical information representations.

In certain embodiments, the semantic transformation module 218 may also resolve variations in prescription data representations that may occur across different healthcare systems. For example, prescription instructions retrieved from different EHR systems or pharmacy dispensing systems may use varying terminologies or formats for representing dosage schedules or medication administration instructions. The semantic transformation module 218 may normalize such variations into a consistent representation within the semantic medication instruction object. In some implementations, the semantic transformation module 218 may also identify relationships between multiple instruction components associated with a medication order. For example, the semantic transformation module 218 may associate dosage frequency with specific administration time intervals or link warning attributes to particular medication administration conditions. Such associations may be represented within the semantic medication instruction object to preserve contextual relationships within the prescription information.

In an embodiment, the semantic medication instruction object generated by the semantic transformation module 218 may serve as an intermediate data structure used by the system 110 for generating patient-specific medication instructions. The normalized semantic representation may allow subsequent processing components to operate on a consistent set of structured instruction attributes regardless of the format or source of the original prescription data.

In an embodiment, the instruction generation module 220 may generate patient-specific medication instructions from the normalized semantic representation generated by the semantic transformation module 218. The instruction generation module 220 may utilize one or more artificial-intelligence-based constrained language generation engines to convert the normalized semantic representation into human-understandable medication instructions suitable for delivery to the user 102.

In an embodiment, the instruction generation module 220 may receive the semantic medication instruction object representing prescription attributes such as dosage frequency, administration route, treatment duration, contraindications, and warning attributes. The instruction generation module 220 may process the semantic medication instruction object together with accessibility parameters associated with the patient profile to generate medication instructions tailored to the needs of the user 102.

In certain implementations, the artificial-intelligence-based constrained language generation engines may apply language translation operations to generate medication instructions consistent with the preferred language associated with the patient profile. The preferred language parameter may indicate a language selected by the user 102 or recorded in the patient profile within the EHR data source 108. In some embodiments, the artificial-intelligence-based constrained language generation engines may apply literacy-level simplification rules based on the literacy level indicator associated with the patient profile. Such simplification rules may reduce sentence complexity, substitute technical medical terminology with more easily understandable language, or restructure instructions into simpler phrases suitable for comprehension by users with lower literacy levels. In certain implementations, the instruction generation module 220 may adapt the generated medication instructions based on the visual impairment indicator associated with the patient profile. For example, the generated instructions may be structured to facilitate delivery through non-textual formats such as audio instructions or simplified visual guidance. In certain embodiments, the artificial-intelligence-based constrained language generation engines may perform text-to-text transformation of medication instructions from a source language into one or more target languages based on the preferred language associated with the patient profile.

In some embodiments, the artificial-intelligence-based constrained language generation engines may also adapt generated instructions based on the dyslexia indicator associated with the patient profile. For example, the generation process may produce instructions that minimize complex sentence structures, avoid visually confusing formatting patterns, or emphasize simplified language constructs. In certain implementations, the instruction generation module 220 may also consider the audio-preference indicator associated with the patient profile. The audio-preference indicator may indicate that the user 102 prefers medication instructions delivered in spoken form rather than text-based instructions. The instruction generation module 220 may therefore generate instruction content suitable for conversion into audio instructions.

In some embodiments, the artificial-intelligence-based constrained language generation engines may generate medication instructions using a dialect variant of the preferred language. The dialect variant may be determined based on regional parameters stored in the patient profile or inferred from language settings associated with the patient profile.

In certain implementations, the constrained language generation engines may operate using predefined linguistic constraints or domain-specific language models configured to ensure that generated medication instructions accurately reflect the semantic medication instruction object. Such constraints may ensure that dosage schedules, treatment duration, contraindications, and warnings are correctly represented in the generated instructions. In an embodiment, the generated patient-specific medication instructions may represent a textual or structured instruction output that describes how the medication should be administered, including dosage timing, administration method, treatment duration, and precautionary information relevant to the prescribed medication. In certain embodiments, such constrained generation may ensure that text-to-text transformed instructions preserve semantic accuracy across languages while remaining suitable for subsequent speech synthesis.

In certain embodiments, the artificial-intelligence-based constrained language generation engines may further include a terminology validation mechanism configured to verify translated medication instructions against standardized medical vocabularies, regulatory-approved medical glossaries, or curated pharmaceutical terminology datasets. Such validation may ensure that translation operations performed across different languages preserve the clinical meaning of prescription instructions, dosage parameters, contraindications, and warning information, thereby preventing semantic distortion of medication guidance generated for the patient.

In some embodiments, the instruction generation module 220 may generate multiple instruction representations suitable for different output formats supported by the system 110. In certain embodiments, the system 110 may further include a human review interface enabling language specialists, pharmacists, or medical translators to review generated medication instructions. Feedback provided by such reviewers may be used to validate linguistic accuracy, confirm medical terminology correctness, or refine language generation models used by the instruction generation module. Such human-in-the-loop validation may improve accuracy of multilingual medication instructions generated by the system.

In an embodiment, the rendering module 222 may render the generated patient-specific medication instructions in one or more output formats accessible to the user 102 through the user device 104. The rendering module 222 may convert the medication instructions generated by the instruction generation module 220 into presentation formats that can be consumed by the user 102 in accordance with accessibility parameters associated with the patient profile.

In an embodiment, the rendering module 222 may generate audio output by providing the generated patient-specific medication instructions, including textual instructions transformed into preferred languages, to a text-to-speech synthesis engine. The text-to-speech synthesis engine may convert the generated instructions into spoken instructions using phonetic conversion and speech synthesis models. The resulting audio instructions may allow the user 102 to listen to medication guidance through speakers, headphones, or other audio interfaces associated with the user device 104. In some embodiments, the rendering module 222 may generate text output representing the medication instructions. The text output may be produced by converting the generated medication instructions into formatted instructional text using structured medical instruction templates. In certain implementations, the text output may be formatted in accordance with accessibility parameters associated with the patient profile. For example, the text output may be rendered in a larger font size or simplified layout to accommodate visual accessibility requirements of the user 102.

In certain embodiments, the rendering module 222 may generate video output representing medication administration instructions. The video output may be generated by a video composition engine configured to assemble a sequence of instructional visual segments representing medication administration steps. For example, the video composition engine may map dosage timing parameters to a temporal sequence of animated visual scenes illustrating medication intake events and associated precautionary information.

In some embodiments, the rendering module 222 may also generate pictographic graphical output representing medication instructions. The pictographic graphical output may be generated by mapping medication administration parameters contained within the generated instructions to a predefined library of dosage and administration pictograms. Such pictographic instructions may allow the user 102 to understand medication guidance through visual representations rather than textual instructions. In certain embodiments, the pictographic graphical output may be generated using standardized healthcare pictogram libraries designed for medication communication. Such pictograms may include internationally recognized medical pictogram sets, including pictograms aligned with World Health Organization (WHO) medication communication standards, thereby improving comprehension of medication instructions among patients with low literacy levels or limited language proficiency.

In certain implementations, the rendering module 222 may produce multiple output formats for the same medication instructions. For example, the system 110 may generate audio instructions, text instructions, pictographic representations, or video-based guidance simultaneously, allowing the user 102 to access the instructions using a preferred format. In certain embodiments, the generated outputs may include both translated textual instructions and corresponding speech outputs derived from the translated text. In some embodiments, the rendering module 222 may also generate a machine-readable encoded access object embedding a reference to the generated patient-specific medication instructions. The machine-readable encoded access object may include at least one of a Quick Response (QR) code, a near-field communication (NFC) element, or an embossed scannable label that may be printed on a medication leaflet or associated with medication packaging.

In certain embodiments, the rendering module 222 may dynamically determine a preferred output format based on contextual environmental conditions associated with the user device 104. For example, ambient noise levels detected through device microphones may cause the system 110 to prioritize visual or textual instruction formats over audio output. Similarly, connectivity status or device resource conditions may influence whether instructions are streamed from the system or retrieved from locally stored content. Such adaptive modality selection may improve accessibility and comprehension of medication instructions under varying user environments.

In certain implementations, the machine-readable encoded access object may include an encoded reference identifier corresponding to the generated medication instructions. The encoded reference identifier may allow the user device 104 to retrieve or access the medication instructions when the machine-readable encoded access object is scanned or tapped by the user 102. In some embodiments, the encoded reference identifier may be generated using an encrypted reference identifier that is encoded within the machine-readable access object. The encrypted reference identifier may include an expiration parameter that limits the duration during which the generated medication instructions may be accessed through the encoded access object.

In certain embodiments, the machine-readable encoded access object may reference instruction content that can be locally cached or downloaded onto the user device 104. Such local storage may enable retrieval of medication instructions in offline mode or in environments with limited network connectivity, thereby allowing patients to access medication guidance even when continuous network communication with the system 110 is unavailable. In certain embodiments, generation and distribution of the machine-readable encoded access objects may incorporate security and access control mechanisms to ensure that retrieval of medication instructions complies with healthcare information governance requirements associated with patient-specific medical information.

In an embodiment, the rendering module 222 may provide the rendered medication instructions or references associated with the machine-readable encoded access object to the user device 104 through the communication network 106. The user 102 may then access the rendered medication instructions through the user device 104 in accordance with the preferred output format.

In an embodiment, presentation of the generated medication instructions may be adapted in accordance with accessibility communication guidelines used within healthcare environments to ensure that the instructions remain accessible and understandable to patients with diverse accessibility requirements. Such adaptations may apply across one or more output modalities, including textual instructions, audio narration, visual guidance, video demonstrations, and pictographic representations, thereby supporting patients with visual impairments, cognitive accessibility needs, low literacy levels, or other accessibility considerations.

In an embodiment, the update listener module 224 may detect modification of one or more prescription parameters associated with a medication order stored in the EHR data source 108. The update listener module 224 may monitor the EHR data source 108 or other healthcare information systems for changes to prescription records associated with the patient profile corresponding to the user 102.

In certain implementations, the update listener module 224 may detect modifications to prescription parameters including, but not limited to, dosage modifications, medication formulation changes, addition of contraindication warnings, modification of administration instructions, or medication discontinuation events. Such modifications may occur when a healthcare provider updates the prescription information within the EHR system or when pharmacy dispensing systems record changes to medication orders. In some embodiments, the update listener module 224 may monitor prescription records using event-driven mechanisms or periodic synchronization with the EHR data source 108. For example, the update listener module 224 may receive notification events generated by the EHR system when prescription parameters associated with a medication order are modified. In other implementations, the update listener module 224 may periodically query the EHR data source 108 to determine whether prescription records associated with the patient profile have been updated.

In an embodiment, upon detecting a modification to one or more prescription parameters, the update listener module 224 may trigger regeneration of patient-specific medication instructions by the system 110. The regeneration process may involve retrieving updated prescription data, performing sanitization and extraction operations, transforming the updated prescription data into a normalized semantic representation, and generating updated medication instructions. In certain implementations, the update listener module 224 may also cause previously generated references associated with the medication instructions to be invalidated when prescription parameters change. For example, previously generated machine-readable encoded access objects, such as QR codes or NFC references associated with earlier medication instructions, may be invalidated to prevent access to outdated medication guidance.

In some embodiments, the update listener module 224 may generate updated references or encoded access objects corresponding to the regenerated medication instructions. The updated references may allow the user 102 to access the latest medication instructions through the user device 104. In certain embodiments, the update listener module 224 may maintain records of prescription updates and corresponding instruction regeneration events. Such records may allow the system 110 to track changes in prescription instructions over time and ensure that the most recent medication guidance is provided to the user 102.

In certain embodiments, the other module(s) 226 may perform additional processing functions associated with generation, management, delivery, or monitoring of patient-specific medication instructions. The other module(s) 226 may include auxiliary components configured to support operations of the modules 206 described herein. For example, the other module(s) 226 may include modules configured to manage system configuration parameters, maintain audit logs associated with medication instruction generation events, manage user interaction events, support integration with external healthcare systems, or perform data validation and monitoring operations. In some embodiments, the other module(s) 226 may also support future enhancements to the system 110, including incorporation of additional instruction delivery modalities, integration with emerging healthcare data interoperability standards, or incorporation of additional decision-support mechanisms related to medication administration guidance. In certain embodiments, the other module(s) 226 may include module(s) to record patient interaction events associated with access to medication instructions. Such interaction events may include scanning of QR codes, tapping of near-field communication elements, playback of audio instructions, viewing of video instructions, or other instruction access events initiated through the user device 104. The recorded interaction data may be used to analyze patient engagement with medication instructions and may support compliance monitoring or healthcare provider review. The other module(s) 226 may operate independently or in coordination with the processors 202 and the modules 206 to extend functionality of the system 110.

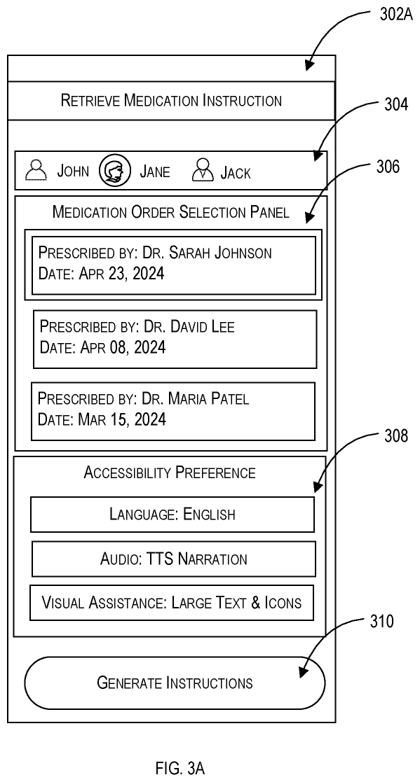

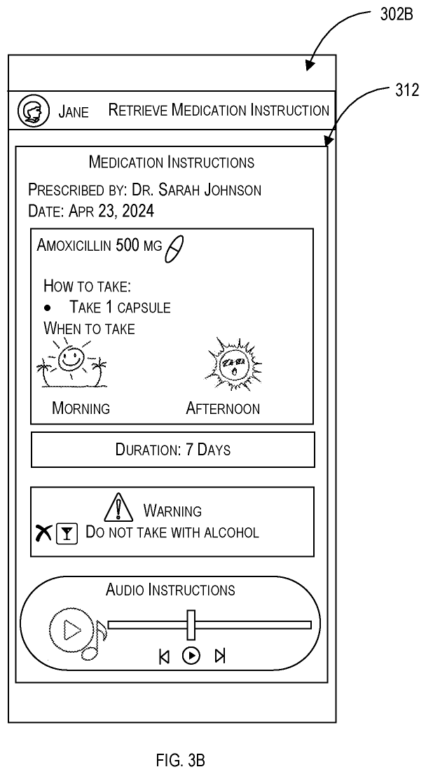

FIG. 3A-3B illustrate exemplary multimedia medication instruction interfaces 302A and 302B presented on the user device 104 for displaying patient-specific medication instructions, in accordance with an embodiment of the present disclosure. For the sake of brevity, FIG. 3A-3B has been explained together.

In an embodiment, the user interface 302A displayed on the user device 104 may facilitate the user 102 to initiate retrieval and generation of patient-specific medication instructions. In an embodiment, the user interface 302A may be presented through an application executed on the user device 104 or through a web-based interface accessible through the user device.

In certain embodiments, the interface 302A may include a patient selection panel allowing the user 102 to select a patient profile associated with the medication instructions, as shown by 304. For example, the panel may present selectable patient profile identifiers such as user icons or names (e.g., John, Jane, Jack). Selection of a patient profile may facilitate the system 110 to identify the corresponding patient profile from which prescription information and accessibility parameters are to be retrieved.

Further, the interface 302A may include a medication order selection panel, as shown by 306, configured to display one or more medication orders associated with the selected patient profile. Each medication order may display identifying information, such as the prescribing healthcare provider and the date on which the prescription was issued. The user 102 may select a medication order from the medication order selection panel to initiate retrieval of structured prescription data corresponding to the selected medication order. In some embodiments, the interface 302A may further include an accessibility preference section, as shown by 308, through which accessibility parameters associated with the patient profile may be displayed or selected. The accessibility preference section may include settings such as preferred language, audio narration preferences, or visual assistance options. These accessibility parameters may correspond to the accessibility parameters retrieved by the system 110 and used during generation of the patient-specific medication instructions.

In certain embodiments, the interface 302A may further include an instruction generation control, as shown by 310, that facilitates the user 102 to initiate generation of medication instructions. For example, the instruction generation control may be represented as a selectable interface element such as a “Generate Instructions” button. Upon activation of the instruction generation control, the user device 104 may transmit a request to the system 110 to retrieve structured prescription data associated with the selected medication order from the EHR data source 108.

In some embodiments, the user interface 302A may further provide visual indicators or status feedback indicating that medication instruction generation has been initiated. Such feedback may inform the user 102 that the medication instructions associated with the selected medication order are being retrieved.

In an embodiment, the interface 302B may include a medication instruction display section that identifies the medication associated with the retrieved medication order, as shown by 312. The medication instruction display section may present the medication name and dosage strength corresponding to the prescription retrieved from the EHR data source 108. For example, the interface 302B may display medication information such as “Amoxicillin 500 mg” associated with the medication order selected by the user 102. In some embodiments, the medication instruction display section may further present visual characteristics of the medication to assist the user 102 in identifying the prescribed medication. Such visual characteristics may include the shape, color, texture, or other physical attributes of the medication, which may be displayed as graphical representations or images corresponding to the prescribed medication.

In certain embodiments, the medication instruction display section, shown by 312 may further display administration instructions describing how the medication should be taken. The administration instructions may include dosage instructions generated by the system 110 based on the normalized semantic representation of the prescription data. For example, the instructions may specify that the user 102 should take a specified quantity of the medication, such as “Take 1 capsule”, in accordance with the dosage parameters extracted from the prescription record.

In some embodiments, the medication instruction display section, shown by 312, may also present medication administration timing information indicating when the medication should be taken. The timing information may include visual indicators representing specific times of day, such as morning, afternoon, or evening, corresponding to the dosage frequency specified within the prescription data. Such timing indicators may provide a simplified visual representation of the medication schedule to improve comprehension by the user.

In an embodiment, the medication instruction display section, shown by 312, may further include a duration indicator representing the prescribed treatment duration associated with the medication order. For example, the medication instruction display section, shown by 312, may indicate a treatment duration such as “7 days”, which corresponds to the treatment duration parameter extracted from the structured prescription data. In certain embodiments, the medication instruction display section, shown by 312, may also include a warning or precaution section presenting safety information associated with the prescribed medication. The warning section may include contraindications, precautionary statements, or administration guidelines derived from the prescription record. For example, the warning section may indicate that the medication should not be taken with alcohol or that the medication should be taken after food.

In some embodiments, the medication instruction display section, shown by 312, may further include an audio instruction playback control that enables the user 102 to listen to spoken medication instructions generated by the system 110. The audio instruction playback control may allow the user 102 to initiate playback of medication instructions produced using a text-to-speech synthesis engine. Such audio instructions may provide an alternative modality for presenting medication guidance, particularly for users with accessibility preferences for audio-based instruction delivery.

In certain implementations, the interface 302B may combine textual instruction elements with graphical icons or visual indicators representing medication administration steps, timing indicators, or warnings. These visual elements may support improved comprehension of medication instructions, particularly for users with lower literacy levels or visual learning preferences.

In an embodiment, the interface 302B may be dynamically generated by the system 110 after patient-specific medication instructions are produced by the instruction generation module 220 and rendered by the rendering module 222. The interface 302B may therefore represent one example of how the generated medication instructions may be presented to the user 102 through the user device 104.

In an embodiment, the interface 302B may present medication instructions using multiple output modalities simultaneously, including textual instructions, visual guidance, pictographic dosage indicators, and audio narration. In an embodiment, the interface 302B may include an instruction text panel configured to display textual medication instructions generated by the system 110. The instruction text panel may present medication identification information and dosage instructions derived from the structured prescription data. In certain embodiments, the interface 302B may further include an animated medication administration sequence representing visual guidance for medication intake events. The animated sequence may illustrate medication administration steps corresponding to the dosage timing parameters associated with the medication order. For example, the animated sequence may display a capsule icon moving along a timeline representing different times of day, such as morning, afternoon, and evening. The animated sequence may be generated by a video composition engine that maps dosage timing parameters derived from the semantic medication instruction object to a sequence of visual scenes representing medication administration events. In certain embodiments, the textual instructions presented within the interface may be generated through text-to-text transformation into a preferred language and synchronized with corresponding speech output generated from the transformed text.

In some embodiments, the interface 302B may also present pictographic dosage indicators representing medication intake events through graphical icons. The pictographic dosage indicators may include visual symbols representing time-of-day indicators such as morning, afternoon, or evening. Capsule icons may be aligned with the corresponding time-of-day indicators to visually represent dosage frequency associated with the prescribed medication. Such pictographic representations may improve comprehension of medication instructions, particularly for users with lower literacy levels.

In an embodiment, the interface 302B may further include audio narration controls, enabling the user 102 to initiate playback of spoken medication instructions. The audio narration control may allow the user 102 to play or pause audio instructions generated by the system 110 using a text-to-speech synthesis engine. The audio instructions may correspond to the same medication guidance presented in the textual and graphical components of the interface. In certain implementations, the audio narration may be synchronized with the animated medication administration sequence, allowing spoken instructions to correspond with the visual guidance displayed within the interface. Such synchronization may enable coordinated audio-visual presentation of medication instructions.

In some embodiments, the interface 302B may also incorporate accessibility-adapted display parameters, such as large text formatting, simplified language structures, or enhanced visual icons. These accessibility-adapted parameters may be applied based on accessibility parameters associated with the patient profile retrieved by the system 110. In an embodiment, the multimedia medication instruction interface 302B may present multiple instruction formats concurrently, allowing the user 102 to understand medication guidance through textual, visual, and auditory channels. In certain embodiments, the visual elements and pictographic representations may be selected or rendered in accordance with standardized healthcare communication guidelines, including globally recognized pictogram standards, to ensure consistent interpretation across diverse patient groups.

In some embodiments, the medication instructions described herein may correspond to different dosage forms, including solid, liquid, and gaseous medications. For example, solid medications may include tablets, capsules, or coated pills, and the instructions may indicate dosage quantity, intake frequency, and timing of administration. Liquid medications may include syrups, suspensions, or oral solutions, and the instructions may indicate dosage volume, such as milliliters or teaspoons, along with guidance for measuring the prescribed quantity using a dosing cup, measuring spoon, or syringe. In certain embodiments, gaseous medications may include inhalable medications administered through inhalers, nebulizers, or similar delivery devices, and the instructions may indicate inhalation dosage, number of inhalations, timing intervals, and usage procedures associated with the inhalation device.

In an embodiment, the generated medication instructions may adapt the presented guidance based on the dosage form associated with the prescribed medication. For solid medications, such as tablets, capsules, or coated pills, the instructions may indicate the number of units to be taken per dose, the frequency of administration during a day, timing relative to meals or other activities, and precautions such as swallowing the medication whole, chewing where applicable, or avoiding crushing in certain cases. The instructions may further include visual or pictographic indicators representing the number of tablets or capsules to be taken at each scheduled administration time. For liquid medications, such as syrups, suspensions, or oral solutions, the instructions may indicate a dosage volume expressed in units such as milliliters, teaspoons, or other standardized measures. The instructions may further provide guidance for measuring the prescribed dosage using a dosing cup, calibrated syringe, dropper, or measuring spoon. In some embodiments, the instructions may also indicate preparatory steps such as shaking the bottle prior to administration, maintaining the medication at a recommended storage condition, or ensuring accurate measurement of the prescribed volume. For gaseous medications, such as inhalable medications administered using inhalers, nebulizers, or other inhalation delivery devices, the instructions may indicate the number of inhalations to be taken per dose, timing intervals between inhalations, and the recommended frequency of administration during a prescribed treatment period. The instructions may further include usage guidance associated with the inhalation device, such as shaking the inhaler before use, coordinating inhalation with actuation of the device, or maintaining a specified breathing pattern during inhalation. Visual or animated guidance may also be presented to demonstrate proper usage of the inhalation device to ensure effective medication delivery.

In an embodiment, the generated medication instructions may include references to external instructional resources accessible through network-based links. Such references may direct the user 102 to instructional content hosted on online platforms or medical information websites. For example, the instructions may include hyperlinks, embedded media links, or scannable references that allow the user 102 to access demonstration videos or instructional content illustrating proper medication administration techniques, device usage procedures, or other guidance associated with the prescribed medication. In some embodiments, such external instructional resources may be presented in a language consistent with the preferred language associated with the patient profile.

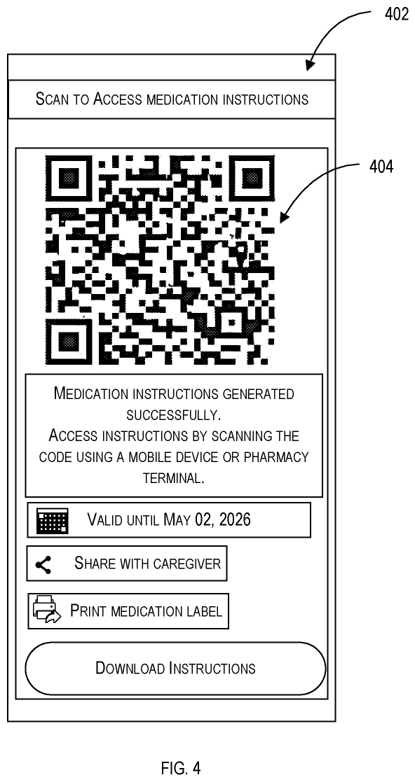

FIG. 4 illustrates an exemplary interface 402 presented on the user device 104 for enabling retrieval of patient-specific medication instructions through the machine-readable encoded access object, in accordance with an embodiment of the present disclosure.

In an embodiment, the interface 402 may include a machine-readable encoded access object 404 representing a reference to the patient-specific medication instructions generated by the system 110. The machine-readable encoded access object 404 may be displayed as a Quick Response (QR) code or other scannable encoded object that can be scanned by a compatible device, such as a mobile device, pharmacy terminal, or other computing system capable of decoding the encoded reference.

In certain embodiments, the machine-readable encoded access object 404 may encode a reference identifier corresponding to the generated medication instructions. The reference identifier may allow the user device 104 or another device to retrieve the corresponding medication instructions from the system 110 when the encoded object is scanned or accessed. In some implementations, the encoded reference identifier may be generated as an encrypted reference identifier. The encrypted reference identifier may protect access to the medication instructions by preventing unauthorized decoding or modification of the encoded reference.

In certain embodiments, the interface 402 may further display an instruction access confirmation message indicating that the medication instructions have been successfully generated by the system 110. The confirmation message may inform the user 102 that the instructions can be accessed by scanning the machine-readable encoded access object using a compatible device.

In some embodiments, the interface 402 may include an access validity or expiration indicator associated with the encoded access object. The expiration indicator may display a validity period during which the encoded reference identifier may be used to access the medication instructions. For example, the interface may indicate a date until which the encoded access object remains valid. Such expiration parameters may prevent retrieval of outdated medication instructions after prescription information has been modified. In certain implementations, the interface 402 may further include sharing or printing controls that enable the user 102 to distribute or store the encoded medication instruction reference. For example, the sharing control may allow the user 102 to share the encoded access object with a caregiver or other authorized individual, while the printing control may enable printing of a medication label or leaflet containing the encoded access object. In some embodiments, the interface 402 may also provide additional controls enabling the user 102 to download or locally store the medication instructions associated with the encoded access object. These controls may allow the user 102 to access the instructions at a later time without requiring immediate retrieval from the system 110.

In an embodiment, the machine-readable encoded access object 404 may enable convenient retrieval of the instructions, while the expiration indicator and encrypted reference identifier may ensure that the accessed instructions correspond to the most current prescription information stored within the system 110.

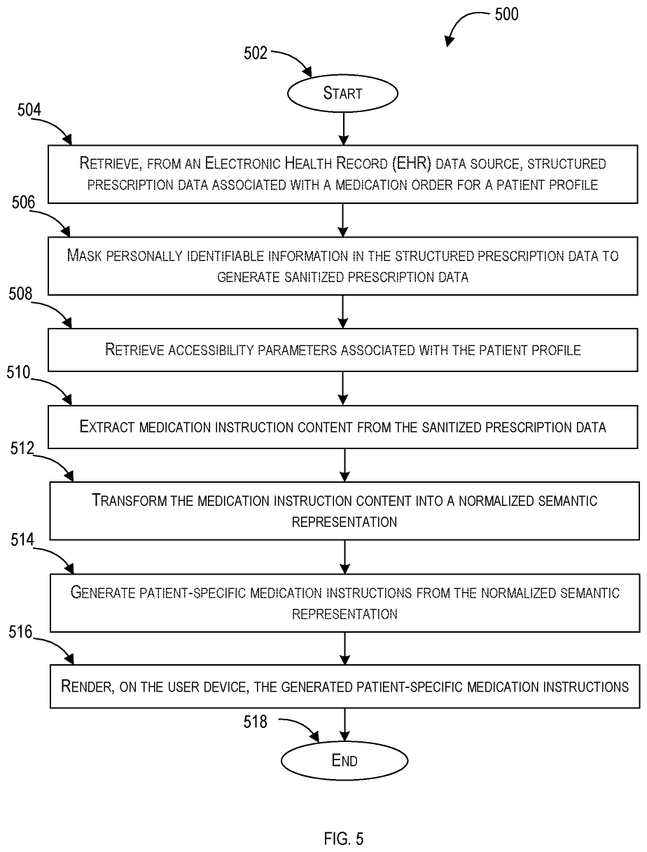

FIG. 5 illustrates an exemplary flowchart 500 of a method for generating and delivering patient-specific medication instructions, in accordance with an embodiment of the present disclosure. The method may be initiated in response to a request generated by a user through a user device to obtain medication instructions corresponding to a medication order associated with a patient profile. The method starts at step 502.

At step 504, the method includes retrieving, from an Electronic Health Record (EHR) data source, structured prescription data associated with a medication order for a patient profile, wherein the structured prescription data is retrieved in response to a request initiated by a user through a user device. The structured prescription data may include medication identifiers, dosage instructions, administration routes, frequency schedules, treatment duration, contraindications, and warning information associated with the medication order.

At step 506, the method includes masking personally identifiable information in the structured prescription data to generate sanitized prescription data without personally identifiable information. Masking may include replacing personally identifiable information fields in the structured prescription data with anonymized tokens to produce the sanitized prescription data.

At step 508, the method includes retrieving accessibility parameters associated with the patient profile, the accessibility parameters comprising at least one of a preferred language, a literacy level indicator, a visual impairment indicator, a dyslexia indicator, and an audio-preference indicator.

At step 510, the method includes extracting medication instruction content from the sanitized prescription data. Extraction of the medication instruction content may include performing structured data parsing of prescription fields, including dosage instructions, administration routes, frequency schedules, treatment duration, contraindications, and warning information.

At step 512, the method includes transforming the medication instruction content into a normalized semantic representation, wherein the normalized semantic representation comprises a standardized semantic medication instruction object including dosage frequency, administration route, duration, contraindications, and warning attributes.

At step 514, the method includes generating patient-specific medication instructions from the normalized semantic representation based on the retrieved accessibility parameters using one or more artificial-intelligence-based constrained language generation engines. Generation of the patient-specific medication instructions may include performing text-to-text transformation of medication instruction content into one or more target languages consistent with the preferred language, applying literacy-level simplification rules based on the literacy level indicator, adapting the generated instructions based on the visual impairment indicator, adapting the generated instructions based on the dyslexia indicator, adapting the generated instructions based on the audio-preference indicator, and generating the instructions using a dialect variant of the preferred language determined from regional parameters stored in the patient profile. In certain embodiments, the generated textual instructions may be further processed to produce corresponding speech representations using text-to-speech synthesis. In certain embodiments, generation and delivery of the patient-specific medication instructions may be performed in accordance with healthcare regulatory compliance frameworks governing patient information handling, translation accuracy, and accessibility of medical information.

At step 516, the method includes rendering, on the user device, the generated patient-specific medication instructions in at least one output format. The output format may comprise text output and audio output. The audio output being generated by a text-to-speech synthesis engine configured to convert the generated patient-specific medication instructions, including textual instructions generated consistent with the preferred language, into spoken instructions using phonetic conversion and speech synthesis models. In certain embodiments, the output formats may include text output generated using structured medical instruction templates, video output generated by a video composition engine configured to assemble instructional visual segments representing medication administration steps, and pictographic graphical output generated by mapping medication administration parameters to a predefined library of dosage and administration pictograms. In certain embodiments, generation and rendering of the patient-specific medication instructions may be performed under regulatory compliance constraints, ensuring that translated instructions, dosage representations, and warning information remain consistent with standardized medical communication references used in healthcare systems.

In an embodiment, the method further includes synchronizing synthesized speech with animated graphical overlays during presentation of video-based medication instructions. Such synchronization may allow spoken instructions generated by the text-to-speech synthesis engine to correspond with visual guidance representing medication administration events.

In an embodiment, the method further includes generating a machine-readable encoded access object embedding a reference to the generated patient-specific medication instructions. The machine-readable encoded access object may comprise at least one of a Quick Response (QR) code, a near-field communication element, or an embossed scannable label that enables retrieval of the generated medication instructions. In an embodiment, the machine-readable encoded access object may be generated using an encrypted reference identifier, the encrypted reference identifier being encoded into the machine-readable encoded access object. The encrypted reference identifier may further include an expiration parameter limiting access validity to a predefined duration.

In an embodiment, the method may further include detecting modification of one or more prescription parameters in the Electronic Health Record data source. The modification may comprise at least one of a dosage modification, a medication formulation change, an added contraindication warning, or a medication discontinuation event. In an embodiment, detection of the modification may trigger regeneration of the patient-specific medication instructions and invalidation of previously generated machine-readable encoded access objects, thereby ensuring that retrieved medication instructions correspond to updated prescription information. The method ends at step 518.

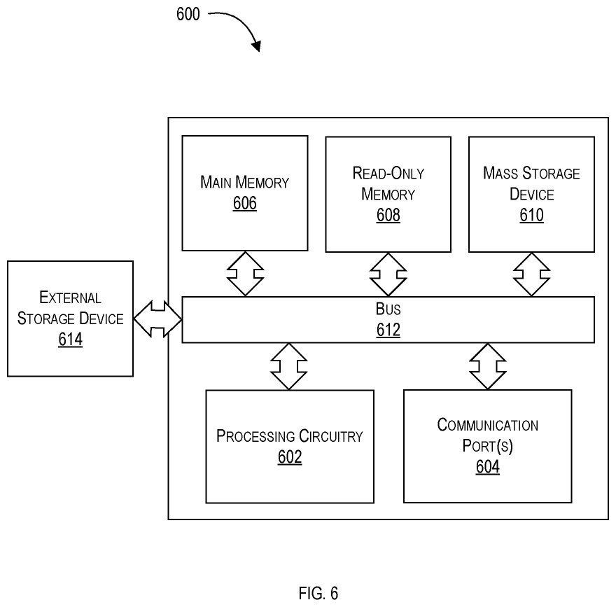

FIG. 6 illustrates an exemplary computer system in which or with which embodiments of the present disclosure may be utilized. As shown in FIG. 6, a computer system 600 includes an external storage device 614, a bus 612, a main memory 606, a read-only memory 608, a mass storage device 610, a communication port 604, and a processor 602.

Those skilled in the art will appreciate that computer system 600 may include more than one processor 602 and communication ports 604. Examples of processor 602 include, but are not limited to, an Intel® Itanium® or Itanium 2 processor(s), or AMD® Opteron® or Athlon MP® processor(s), Motorola® lines of processors, FortiSOC™ system on chip processors, or other future processors. The processor 602 may include various modules associated with embodiments of the present disclosure.

The communication port 604 can be any of an RS-232 port for use with a modem-based dialup connection, a 10/100 Ethernet port, a Gigabit or 10 Gigabit port using copper or fiber, a serial port, a parallel port, or other existing or future ports. The communication port 604 may be chosen depending on a network, such as a Local Area Network (LAN), Wide Area Network (WAN), or any network to which the computer system connects.

The memory 606 can be Random Access Memory (RAM), or any other dynamic storage device commonly known in the art. Read-Only Memory 608 can be any static storage device(s), e.g., but not limited to, a Programmable Read-Only Memory (PROM) chip for storing static information, e.g., start-up or BIOS instructions for processor 602. In certain embodiments, the computing infrastructure used to implement the system may operate within secure healthcare processing environments configured to enforce healthcare data protection policies, access control mechanisms, and secure data handling procedures applicable to patient health information.

The mass storage 610 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7200 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

The bus 612 communicatively couples the processor(s) 602 with the other memory, storage, and communication blocks. The bus 612 can be, e.g., a Peripheral Component Interconnect (PCI)/PCI Extended (PCI-X) bus, Small Computer System Interface (SCSI), USB, or the like, for connecting expansion cards, drives, and other subsystems as well as other buses, such a front side bus (FSB), which connects processor 602 to a software system.

Optionally, operator and administrative interfaces, e.g., a display, keyboard, and a cursor control device, may also be coupled to bus 612 to support direct operator interaction with the computer system. Other operator and administrative interfaces can be provided through network connections connected through communication port 604. The external storage device 614 can be any kind of external hard drives, floppy drives, IOMEGA® Zip Drives, Compact Disc-Read-Only Memory (CD-ROM), Compact Disc-Re-Writable (CD-RW), Digital Video Disk Read Only Memory (DVD-ROM). The components described above are meant only to exemplify various possibilities. In no way should the aforementioned exemplary computer system limit the scope of the present disclosure.

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

Thus, it will be appreciated by those of ordinary skill in the art that the diagrams, schematics, illustrations, and the like represent conceptual views or processes illustrating systems and methods embodying this disclosure. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and, thus, are not intended to be limited to any particular named.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices can exchange data with each other over the network, possibly via one or more intermediary devices.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, utilized, or combined with other elements, components, or steps that are not expressly referenced. Where the specification claims refer to at least one of something selected from the group consisting of A, B, C, . . . , and N, the text should be interpreted as requiring only one element from the group, not A plus N, or B plus N, etc.

While the foregoing describes various embodiments of the invention, other and further embodiments of the invention may be devised without departing from the basic scope thereof. The scope of the invention is determined by the claims that follow. The invention is not limited to the described embodiments, versions, or examples, which are included to enable a person having ordinary skill in the art to make and use the invention when combined with information and knowledge available to the person having ordinary skill in the art.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded to the widest scope consistent with the principles and novel features disclosed herein. It is contemplated that additional embodiments are within the spirit and true scope of the disclosed subject matter.

What is claimed is:

1. A system for generating and delivering patient-specific medication instructions, the system comprising:
a data retrieval module configured to retrieve, from an Electronic Health Record (EHR) data source, structured prescription data associated with a medication order for a patient profile, wherein the structured prescription data is retrieved in response to a request initiated by a user through a user device;
a data sanitization module configured to mask personally identifiable information in the structured prescription data to generate sanitized prescription data without personally identifiable information;
a patient profile retrieval module configured to retrieve accessibility parameters associated with the patient profile, the accessibility parameters comprising at least one of: a preferred language, a literacy level indicator, a visual impairment indicator, a dyslexia indicator, and an audio-preference indicator;
an instruction extraction module configured to extract medication instruction content from the sanitized prescription data;
a semantic transformation module configured to transform the medication instruction content into a normalized semantic representation, wherein the normalized semantic representation comprises a standardized semantic medication instruction object including at least one of: dosage frequency, administration route, duration, contraindications, and warning attributes;
an instruction generation module configured to generate patient-specific medication instructions from the normalized semantic representation, based on the retrieved accessibility parameters, using one or more artificial-intelligence-based constrained language generation engines;
a rendering module configured to render, on the user device, the generated patient-specific medication instructions in at least one output format, wherein the output format comprises text output and audio output, the audio output being generated by a text-to-speech synthesis engine configured to convert the generated patient-specific medication instructions, including textual instructions consistent with the preferred language, into spoken instructions using phonetic conversion and speech synthesis models.

2. The system as claimed in claim 1, wherein the data sanitization module masks personally identifiable information by replacing personally identifiable information fields in the structured prescription data with anonymized tokens to generate the sanitized prescription data.

3. The system as claimed in claim 1, wherein the instruction extraction module extracts the medication instruction content from the sanitized prescription data using structured data parsing of prescription fields comprising at least one of: dosage, administration route, frequency, duration, contraindications, and warning information.

4. The system as claimed in claim 1, wherein the one or more artificial-intelligence-based constrained language generation engines perform at least one of:
applying language translation consistent with the preferred language, applying literacy-level simplification rules based on the literacy level indicator;
generating textual instructions in the preferred language languages based on the retrieved accessibility parameters;
adapting the generated patient-specific medication instructions based on the visual impairment indicator;
adapting the generated patient-specific medication instructions based on the dyslexia indicator;
adapting the generated patient-specific medication instructions based on the audio-preference indicator; and
generating the patient-specific medication instructions using a dialect variant of the preferred language determined from regional parameters stored in the patient profile.

5. The system as claimed in claim 1,
wherein the output format further comprises text output generated by converting the generated patient-specific medication instructions into formatted instructional text using a structured medical instruction template, the text output being rendered in a font size determined based on the visual impairment indicator associated with the patient profile;
wherein the output format further comprises video output generated by a video composition engine configured to assemble a sequence of instructional visual segments based on prescription dosage parameters, wherein the video composition engine maps dosage timing parameters to a temporal sequence of animated visual scenes representing medication administration events;
wherein the output format further comprises pictographic graphical output generated by mapping medication administration parameters to a predefined library of dosage and administration pictograms.

6. The system as claimed in claim 5, further comprising an audiovisual synchronization controller configured to synchronize synthesized speech with animated graphical overlays.

7. The system as claimed in claim 5, wherein the output format comprises a machine-readable encoded access object embedding a reference to the generated patient-specific medication instructions, wherein the machine-readable encoded access object comprises at least one of: a QR code, a near-field communication element, an embossed scannable label.

8. The system as claimed in claim 1, wherein the machine-readable encoded access object is generated using an encrypted reference identifier, the encrypted reference identifier being encoded into the machine-readable encoded access object and including an expiration parameter limiting access validity to a predefined duration.

9. The system as claimed in claim 1, further comprising an update listener module configured to detect modification of at least one prescription parameter in the EHR data source, wherein the modification comprises at least one of: a dosage modification, a medication formulation change, an added contraindication warning, and a medication discontinuation event.

10. The system as claimed in claim 9, wherein detection of the modification by the update listener module triggers regeneration of the patient-specific medication instructions and invalidation of previously generated machine-readable encoded access objects.

11. A method for generating and delivering patient-specific medication instructions, the method comprising:
retrieving, from an Electronic Health Record (EHR) data source, structured prescription data associated with a medication order for a patient profile, wherein the structured prescription data is retrieved in response to a request initiated by a user through a user device;
masking personally identifiable information in the structured prescription data to generate sanitized prescription data without personally identifiable information;
retrieving accessibility parameters associated with the patient profile, the accessibility parameters comprising at least one of: a preferred language, a literacy level indicator, a visual impairment indicator, a dyslexia indicator, and an audio-preference indicator;
extracting medication instruction content from the sanitized prescription data;
transforming the medication instruction content into a normalized semantic representation, wherein the normalized semantic representation comprises a standardized semantic medication instruction object including at least one of: dosage frequency, administration route, duration, contraindications, and warning attributes;
generating patient-specific medication instructions from the normalized semantic representation, based on the retrieved accessibility parameters, using one or more artificial-intelligence-based constrained language generation engines;
rendering, on the user device, the generated patient-specific medication instructions in at least one output format, wherein the output format comprises text output and audio output, the audio output being generated by a text-to-speech synthesis engine configured to convert the generated patient-specific medication instructions, including textual instructions consistent with the preferred language, into spoken instructions using phonetic conversion and speech synthesis models.

12. The method as claimed in claim 11, comprises masking personally identifiable information by replacing personally identifiable information fields in the structured prescription data with anonymized tokens to generate the sanitized prescription data.

13. The method as claimed in claim 11, comprises extracting the medication instruction content from the sanitized prescription data using structured data parsing of prescription fields comprising at least one of: dosage, administration route, frequency, duration, contraindications, and warning information.

14. The method as claimed in claim 11, wherein the one or more artificial-intelligence-based constrained language generation engines perform at least one of:
applying language translation consistent with the preferred language, applying literacy-level simplification rules based on the literacy level indicator;
generating textual instructions in the preferred language languages based on the retrieved accessibility parameters;
adapting the generated patient-specific medication instructions based on the visual impairment indicator;
adapting the generated patient-specific medication instructions based on the dyslexia indicator;
adapting the generated patient-specific medication instructions based on the audio-preference indicator; and
generating the patient-specific medication instructions using a dialect variant of the preferred language determined from regional parameters stored in the patient profile.

15. The method as claimed in claim 11,
wherein the output format further comprises text output generated by converting the generated patient-specific medication instructions into formatted instructional text using a structured medical instruction template, the text output being rendered in a font size determined based on the visual impairment indicator associated with the patient profile;
wherein the output format further comprises video output generated by a video composition engine configured to assemble a sequence of instructional visual segments based on prescription dosage parameters, wherein the video composition engine maps dosage timing parameters to a temporal sequence of animated visual scenes representing medication administration events;
wherein the output format further comprises pictographic graphical output generated by mapping medication administration parameters to a predefined library of dosage and administration pictograms.

16. The method as claimed in claim 15, further comprising synchronizing synthesized speech with animated graphical overlays using an audiovisual synchronization controller.

17. The method as claimed in claim 15, wherein the output format comprises a machine-readable encoded access object embedding a reference to the generated patient-specific medication instructions, wherein the machine-readable encoded access object comprises at least one of: a QR code, a near-field communication element, an embossed scannable label.

18. The method as claimed in claim 11, wherein the machine-readable encoded access object is generated using an encrypted reference identifier, the encrypted reference identifier being encoded into the machine-readable encoded access object and including an expiration parameter limiting access validity to a predefined duration.

19. The method as claimed in claim 11, further comprising detecting modification of at least one prescription parameter in the EHR data source, wherein the modification comprises at least one of: a dosage modification, a medication formulation change, an added contraindication warning, and a medication discontinuation event.

20. The method as claimed in claim 19, wherein detection of the modification by the update listener module triggers regeneration of the patient-specific medication instructions and invalidation of previously generated machine-readable encoded access objects.
